(19) United States
(12) Patent Application Publication
(10) Pub. No.: US 2008/0085313 A1
(43) Pub. Date: Apr. 10, 2008

(54) Methods and compositions for treatment of sleep apnea

(76) Inventors: Bruce D. Given, Pearland, TX (US); Richard A.F. Dixon, Houston, TX (US)

Correspondence Address: JONES DAY, 222 EAST 41ST ST, NEW YORK, NY, 10017, US

(21) Appl. No.: 11/804,052

(22) Filed: May 15, 2007

Related U.S. Application Data
(60) Provisional application No. 60/800,721, filed on May 15, 2006.

(51) Int. Cl. A61K 9/30; A61K 31/42; A61P 11/00

(52) U.S. Cl. 424/475; 514/380

(57) ABSTRACT

Provided herein are methods of treatment of sleep apnea by administering an endothelin antagonist, such as sitaxsentan or a pharmaceutically acceptable salt thereof to a patient in need of the treatment.

### RELATED APPLICATIONS

This application claims priority to U.S. provisional application Ser. No. 60/800,721, filed May 15, 2006, entitled “METHODS AND COMPOSITIONS FOR TREATMENT OF SLEEP APNEA” to Given et al. The disclosure of the above referenced application is incorporated by reference herein.

### FIELD

Provided herein are methods for treatment of sleep apnea, including, obstructive sleep apnea (OSA) and central sleep apnea (CSA) by administering an endothelin antagonist or a pharmaceutically acceptable salt thereof to a patient in need thereof.

### BACKGROUND

Apnea is the cessation of breathing. Sleep apnea is defined as repetitive occurrences of cessation or diminution of airflow (apnea or hypopnea during sleep), with consequent oxygen desaturation and arousal. Apnea is the cessation of airflow for at least 10 seconds, while hypopnea is defined as a 30% or greater reduction in airflow, lasting 10 seconds or more with oxygen desaturation or EEG (electroencephalogram) evidence of arousal. Sleep apnea can be obstructive (upper airway blockage despite airflow drive), central (decreased respiratory center output), or mixed. Obstructive sleep apnea (OSA) is characterized by preserved and increased respiratory effort despite partial or complete occlusion of the upper airway. Central sleep apnea (CSA) is the absence of both respiratory efforts and airflow. The most common cause for sleep apnea is airway obstruction. Rarely, sleep apnea is due to primary brain stem medullary failure caused by neurologic medullary depression, which may result from poliomyelitis, tumors of the posterior fossa, or idiopathic failure of central (brain stem) breathing control in which patients may breathe insufficiently or not at all except when fully awake. Mixed apnea starts as central apnea, quickly followed by thoracoabdominal movements and upper airway obstruction. Mixed apnea occurs more often than central but less often than obstructive apnea.

The severity of sleep apnea is defined by the apnea-hypopnea index (AHI), which is the number of apneic and hypopneic events per hour of sleep. OSA is frequently seen in obese individuals even in the absence of other coexisting disease. In contrast, CSA is primarily seen in patients with heart failure, but may also occur in healthy people during normal sleep, especially at altitude. Sleep apnea is a common sleep disorder that affects over twelve million (12,000,000) people in the United States.

Therefore, there is continuing need for developing efficient treatments for sleep apnea.

### SUMMARY

In one embodiment, provided herein are methods for treating sleep apnea by administering a compound that has activity as an endothelin antagonist, such as an endothelin A antagonist. In certain embodiments, methods provided herein encompass administering sitaxsentan or a pharmaceutically acceptable salt thereof to a patient in need of such treatment.

Also provided are articles of manufacture containing packaging material, the endothelin antagonist compound, such as sitaxsentan or a pharmaceutically accepted salt thereof and a label that indicates that the compound, such as sitaxsentan or a pharmaceutically accepted salt thereof is used for treating sleep apnea.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, “sleep apnea” is defined as repetitive occurrences of cessation or diminution of airflow (apnea or hypopnea during sleep), with consequent oxygen desaturation and arousal. Sleep apnea can be obstructive (upper airway blockage despite airflow drive), central (decreased respiratory center output), or mixed. Obstructive sleep apnea (OSA) is characterized by preserved and increased respiratory effort despite partial or complete occlusion of the upper airway. Central sleep apnea (CSA) is the absence of both respiratory efforts and airflow.

As used herein, an endothelin agonist is a compound that potentiates or exhibits a biological activity associated with or possessed by an endothelin peptide.

As used herein, an endothelin antagonist is a compound that inhibits endothelin-mediated physiological responses. The antagonist may act by interfering with the interaction of the endothelin with an endothelin-specific receptor or by interfering with the physiological response to or bioactivity of an endothelin isopeptide.

As used herein “sitaxsentan” refers to N-(4-chloro-3-methyl-5-isoxazolyl)-2-[2-methyl-4,5-(methylenedioxy)phenylacetyl]-thiophene-3-sulfonamide. Sitaxsentan is also known as TBC11251. Other chemical names for sitaxsentan include 4-chloro-3-methyl-5-(2-(2-(6-methylbenzo[d][1,3]dioxol-5-yl)acetyl)-3-thienylsulfonamido)isoxazole and N-(4-chloro-3-methyl-5-isoxazolyl)-2-[3,4-(methylenedioxy)-6-methylphenylacetyl]-thiophene-3-sulfonamide. The chemical structures of sitaxsentan and sitaxsentan sodium salt are described elsewhere herein.

As used herein “subject” is an animal, such as a mammal, including human, such as a patient.

As used herein, and unless otherwise specified, the terms “treat,” “treating” and “treatment” contemplate an action that occurs while a patient is suffering from the specified disease or disorder, which reduces the severity of the disease or disorder, or retards or slows the progression of the disease or disorder. Treatment also encompasses any pharmaceutical use of the compositions herein, such as use for treating sleep apnea.

As used herein, amelioration of the symptoms of a particular disorder by administration of a particular pharmaceutical composition refers to any lessening, whether permanent or temporary, lasting or transient that can be attributed to or associated with administration of the composition.

As used herein, unless otherwise specified, the terms “prevent,” “preventing” and “prevention” contemplate an action that occurs before a patient begins to suffer from the specified disease or disorder, which inhibits or reduces the severity of the disease or disorder.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, and unless otherwise specified, the terms “therapeutically effective amount” and “effective amount” of a compound mean an amount sufficient to provide a therapeutic benefit in the treatment, prevent and/or management of a disease, to delay or minimize one or more symptoms associated with the disease or disorder to be treated. The terms “therapeutically effective amount” and “effective amount” can encompass an amount that improves overall therapy, reduces or avoids symptoms or causes of disease or disorder, or enhances the therapeutic efficacy of another therapeutic agent.

As used herein, and unless otherwise specified, the term “prophylactically effective amount” of a compound means an amount sufficient to prevent a disease or disorder, or one or more symptoms associated with the disease or disorder, or prevent its recurrence. The term “prophylactically effective amount” can encompass an amount that improves overall prophylaxis or enhances the prophylactic efficacy of another prophylactic agent.

The terms “co-administration” and “in combination with” include the administration of two therapeutic agents either simultaneously, concurrently or sequentially with no specific time limits. In one embodiment, both agents are present in the cell or in the patient's body at the same time or exert their biological or therapeutic effect at the same time. In one embodiment, the two therapeutic agents are in the same composition or unit dosage form. In another embodiment, the two therapeutic agents are in separate compositions or unit dosage forms.

Methods Of Treatment

Sleep apnea is characterized by repetitive upper airway obstruction with ensuing cyclical hypoxemia, or a decreased level of oxygen in the blood. Hypoxemia induces endothelial cells to produce increased endothelin (see, Kourembanas et al., Hypoxia induces endothelin gene expression and secretion in cultured human endothelium. J Clin Invest. 1991; 88:1054-1057; Rakugi et al., Evidence for endothelin-1 release from resistance vessels of rats in response to hypoxia, Biochem Biophys Res Commun. 1990; 169:973-977; Kanagy et al., Role of endothelin in intermittent hypoxia-induced hypertension, Hypertension 2001, 37, 511-515; Allahdadi et al., Augmented Endothelin Vasoconstriction in intermittent hypoxia-induced hypertension, Hypertension 2005, 45 (part2), 705-709; and Philips et al., Effects of obstructive sleep apnea on endothelin-1 and blood pressure, J. Hypertens 1999, 17:61-66). In the methods provided herein the symptoms associated sleep apnea/hypoapnea are treated, prevented or ameliorated by administering an endothelin antagonist, such as sitaxsentan or a pharmaceutically active derivative thereof. Several endothelin antagonists are known in the art and include, but are not limited to a fermentation product of Streptomyces misakiensis, designated BE-18257B which is a cyclic pentapeptide, cyclo(D-Glu-L-Ala-allo-D-lle-L-Leu-D-Trp); cyclic pentapeptides related to BE-18257B, such as cyclo(D-Asp-Pro-D-Val-Leu-D-Trp) (BQ-123) (see, U.S. Pat. No. 5,114,918 to Ishikawa et al.; see, also, EP A1 0 436 189 to BANYU PHARMACEUTICAL CO., LTD (Oct. 7, 1991)); and other peptide and non-peptidic ETA antagonists have been identified in, for example., U.S. Pat. Nos. 6,432,994; 6,683,103; 6,686,382; 6,248,767; 6,852,745; 5,783,705; 5,962,490; 5,594,021; 5,571,821; 5,591,761; 5,514,691. 5,352,800, 5,334,598, 5,352,659, 5,248,807, 5,240,910, 5,198,548, 5,187,195, 5,082,838, 6,953,780, 6,946,481, 6,852,745, 6,835,741, 6,673,824, 6,670,367, 6,670,362,). These include other cyclic pentapeptides, acyltripeptides, hexapeptide analogs, certain anthraquinone derivatives, indanecarboxylic acids, certain N-pyriminylbenzenesulfonamides, certain benzenesulfonamides, and certain naphthalenesulfonamides (Nakajima et al. (1991) J. Antibiot. 44:1348-1356; Miyata et al. (1992) J. Antibiot. 45:74-8; Ishikawa et al. (1992) J. Med. Chem. 35:2139-2142; U.S. Pat. No. 5,114,918 to Ishikawa et al.; EP A1 0 569 193; EP A1 0 558 258; EP A1 0 436 189 to BANYU PHARMACEUTICAL CO., LTD (Oct. 7, 1991); Canadian Patent Application 2,067,288; Canadian Patent Application 2,071,193; U.S. Pat. No. 5,208,243; U.S. Pat. No. 5,270,313; U.S. Pat. No. 5,612,359, U.S. Pat. No. 5,514,696, U.S. Pat. No. 5,378,715; Cody et al. (1993) Med. Chem. Res. 3:154-162; Miyata et al. (1992) J. Antibiot 45:1041-1046; Miyata et al. (1992) J. Antibiot 45:1029-1040, Fujimoto et al. (1992) FEBS Lett. 305:41-44; Oshashi et al. (1002) J. Antibiot 45:1684-1685; EP A1 0 496 452; Clozel et al. (1993) Nature 365:759-761; International Patent Application WO93/08799; Nishikibe et al. (1993) Life Sci. 52:717-724; and Benigni et al. (1993) Kidney Int. 44:440-444). Numerous sulfonamides that are endothelin peptide antagonists are also described in U.S. Pat. Nos. 5,464,853, 5,594,021, 5,591,761, 5,571,821, 5,514,691, 5,464,853, International PCT application No. 96/31492 and International PCT application No. WO 97/27979.

Further endothelin antagonists described in the following documents, incorporated herein by reference in their entirety, are exemplary of those contemplated for use in methods provided herein: U.S. Pat. No. 5,420,123; U.S. Pat. No. 5,965,732; U.S. Pat. No. 6,080,774; U.S. Pat. No. 5,780,473; U.S. Pat. No. 5,543,521; WO 96/06095; WO 95/08550; WO 95/26716; WO 96/11914; WO 95/26360; EP 601386; EP 633259; U.S. Pat. No. 5,292,740; EP 510526; EP 526708; WO 93/25580; WO 93/23404; WO 96/04905; WO 94/21259; GB 2276383; WO 95/03044; EP 617001; WO 95/03295; GB 2275926; WO 95/08989; GB 2266890; EP 496452; WO 94/21590; WO 94/21259; GB 2277446; WO 95/13262; WO 96/12706; WO 94/24084; WO 94/25013; U.S. Pat. No. 5,571,821; WO 95/04534; WO 95/04530; WO 94/02474; WO 94/14434; WO 96/07653; WO 93/08799; WO 95/05376; WO 95/12611; DE 4341663; WO 95/15963; WO 95/15944; EP 658548; EP 555537; WO 95/05374; WO 95/05372; U.S. Pat. No. 5,389,620; EP 628569; JP 6256261; WO 94/03483; EP 552417; WO 93/21219; EP 436189; WO 96/11927; JP 6122625; JP 7330622; WO 96/23773; WO 96/33170; WO 96/15109; WO 96/33190; U.S. Pat. No. 5,541,186; WO 96/19459; WO 96/19455; EP 713875; WO 95/26360; WO 96/20177; JP 7133254; WO 96/08486; WO 96/09818; WO 96/08487; WO 96/04905; EP 733626; WO 96/22978; WO 96/08483; JP 8059635; JP 7316188; WO 95/33748; WO 96/30358; U.S. Pat. No. 5,559,105; WO 95/35107; JP 7258098; U.S. Pat. No. 5,482,960; EP 682016; GB 2295616; WO 95/26957; WO 95/33752; EP 743307; and WO 96/31492; such as the following compounds described in the recited documents: BQ-123 (Ihara, M., et al., “Biological Profiles of Highly Potent Novel Endothelin Antagonists Selective for the ET_{A} Receptor”, Life Sciences, Vol. 50(4), pp. 247-255 (1992)); PD 156707 (Reynolds, E., et al., “Pharmacological Characterization of PD 156707, an Orally Active ET_{A} Receptor Antagonist”, The Journal of Pharmacology and Experimental Therapeutics, Vol. 273(3), pp. 1410-1417 (1995)); L-754,142 (Williams, D. L., et al., “Pharmacology of L-754,142, a Highly Potent, Orally Active, Nonpeptidyl Endothelin Antagonist”, The Journal of Pharmacology and Experimental Therapeutics, Vol. 275(3), pp. 1518-1526 (1995)); SB 209670 (Ohlstein, E. H., et al., “SB 209670, a rationally designed potent nonpeptide endothelin receptor antagonist”, Proc. Natl. Acad. Sci. USA, Vol. 91, pp. 8052-8056 (1994)); SB 217242 (Ohlstein, E. H., et al., “Nonpeptide Endothelin Receptor Antagonists. VI: Pharmacological Characterization of SB 217242, A Potent and Highly Bioavailable Endothelin Receptor Antagonist”, The Journal of Pharmacology and Experimental Therapeutics, Vol. 276(2), pp. 609-615 (1996)); A-127722 (Opgenorth, T. J., et al., “Pharmacological Characterization of A-127722: An Orally Active and Highly Potent E.sub.TA-Selective Receptor Antagonist”, The Journal of Pharmacology and Experimental Therapeutics, Vol. 276(2), pp. 473-481 (1996)); TAK-044 (Masuda, Y., et al., “Receptor Binding and Antagonist Properties of a Novel Endothelin Receptor Antagonist, TAK-044 {Cyclo[D-α-Aspartyl-3-[(4-Phenylpiperazin-1-yl)Carbonyl]-L-Alanyl-L-α-Aspartyl-D-2-(2-Thienyl)Glycyl-L-Leucyl-D-Tryptophyl]Disodium Salt}, in Human Endothelin_{A} and Endothelin_{B} Receptors”, The Journal of Pharmacology and Experimental Therapeutics, Vol. 279(2), pp. 675-685 (1996)); bosentan (Ro 47-0203, Clozel, M., et al., “Pharmacological Characterization of Bosentan, A New Potent Orally Active Nonpeptide Endothelin Receptor Antagonist”, The Journal of Pharmacology and Experimental Therapeutics, Vol. 270(1), pp. 228-235 (1994)).

In certain embodiments, the endothelin antagonist for use in the methods provided herein is selected from BE-18257B; BQ-123; PD 156707; L-754,142; T-0201; K-8794; PD-156123; PD-156707; PD-160874; PD-180988; S-0139; ZD-1611; BMS-193884; SB 209670; SB 217242; A-127722; TAK-044; tezosentan; bosentan; enrasentan; sitaxsentan and a pharmaceutically acceptable derivative thereof. In one embodiment, provided herein are methods for treatment or amelioration of one or more symptoms of sleep apnea by administering sitaxsentan or a pharmaceutically acceptable salt thereof.

The chemical name for sitaxsentan is N-(4-chloro-3-methyl-5-isoxazolyl)-2-[2-methyl-4,5-(methylenedioxy)phenylacetyl]-thiophene-3-sulfonamide, and its structural formula is as follows:

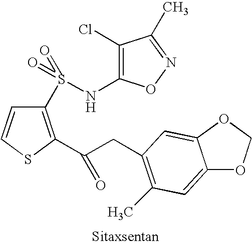

In certain embodiments, the compound for use in the methods provided herein is an alkali metal salt of sitaxsentan. In one embodiment, the compound is sitaxsentan, sodium.

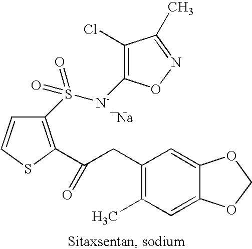

Sitaxsentan sodium is a potent endothelin receptor antagonist that has oral bioavailability in several species, a long duration of action, and high specificity for ETA receptors.

In certain embodiments, sitaxsentan sodium is administered in an amount ranging from about 20 mg up to about 300 mg per day or about 50 mg up to about 300 mg per day. In one embodiment, the amount of sitaxsentan sodium administered is about 25 mg, 50 mg, 60 mg, about 70 mg, 75 mg, about 80 mg, 90 mg, about 100 mg, about 150 mg, about 200 mg, about 250 mg or about 300 mg per day. In one embodiment, the amount of sitaxsentan sodium administered is 50 mg, about 90 mg, about 100 mg or about 150 mg per day. In one embodiment, the amount of sitaxsentan sodium administered is about 100 mg per day.

Methods of Preparation

Sitaxsentan and its sodium salt can be prepared by methods known in the art. An exemplary method for the preparation is described in Example 1. (Also see, U.S. Pat. Nos. 5,783,705, 5,962,490 and 6,248,767).

Pharmaceutical Compositions and Dosage Forms

Pharmaceutical compositions and dosage forms for use in the methods provided herein contain an endothelin antagonist, such as sitaxsentan or sitaxsentan, sodium in a pharmaceutically acceptable carrier and in amounts that are useful in the methods provided herein. Such methods include treatment of sleep apnea, including hypoapnea.

The endothelin antagonist, such as sitaxsentan or sitaxsentan, sodium for use herein is formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. The formulation are prepared using techniques and procedures well known in the art (see, e.g., Ansel Introduction to Pharmaceutical Dosage Forms, Seventh Edition 1999).

In the compositions, effective concentrations of the endothelin antagonist, such as sitaxsentan or sitaxsentan, sodium is (are) mixed with a suitable pharmaceutical carrier or vehicle. The concentration of the endothelin antagonist, such as sitaxsentan or sitaxsentan, sodium in the compositions are effective for delivery of an amount, upon administration, that treats, prevents, or ameliorates one or more of the symptoms of conditions associated with sleep apnea.

In one embodiment, the compositions are formulated for single dosage or multiple dosage administration. To formulate a composition, the weight fraction of the endothelin antagonist, such as sitaxsentan or sitaxsentan sodium is dissolved, suspended, dispersed or otherwise mixed in a selected vehicle at an effective concentration such that the treated condition is relieved or ameliorated. Pharmaceutical carriers or vehicles suitable for administration of the endothelin antagonist, such as sitaxsentan or sitaxsentan, sodium in the methods provided herein include any such carriers known to those skilled in the art to be suitable for the particular mode of administration.

In addition, the endothelin antagonist, such as sitaxsentan or sitaxsentan, sodium may be formulated as the sole pharmaceutically active ingredient in the composition or may be combined with other active ingredients. Liposomal suspensions, including tissue-targeted liposomes, may also be suitable as pharmaceutically acceptable carriers. These may be prepared according to methods known to those skilled in the art. For example, liposome formulations may be prepared as described in U.S. Pat. Nos. 4,522,811; 5,571,534. Briefly, liposomes such as multilamellar vesicles (MLV's) may be formed by drying down egg phosphatidyl choline and brain phosphatidyl serine (7:3 molar ratio) on the inside of a flask. A solution of an active ingredient provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The endothelin antagonist, such as sitaxsentan or sitaxsentan, sodium is included in the pharmaceutically acceptable carrier in an amount sufficient to exert desired effect in the patient treated. The therapeutically effective concentration may be determined empirically by testing the endothelin antagonist, such as sitaxsentan or sitaxsentan, sodium in in vitro and in vivo systems known to one of skill in the art and then extrapolated therefrom for dosages for humans.

The concentration of the endothelin antagonist, such as sitaxsentan or sitaxsentan, sodium in the pharmaceutical composition will depend on absorption, inactivation and excretion rates of the compound, the dosage schedule, and amount administered as well as other factors known to those of skill in the art.

The composition, shape, and type of dosage forms provided herein will vary depending on their use. For example, a dosage form used in the acute treatment of a disease may contain larger amounts of one or more of the active ingredients it contains than a dosage form used in the chronic treatment of the same disease. Similarly, a parenteral dosage form may contain smaller amounts of one or more of the active ingredients it contains than an oral dosage form used to treat the same disease. These and other ways in which specific dosage forms provided herein will vary from one another will be readily apparent to those skilled in the art. See, e.g., Remington's Pharmaceutical Sciences, 20th ed., Mack Publishing, Easton Pa. (2000).

In certain embodiments, the therapeutically effective dosage produces a serum concentration of active ingredient of from about 0.1 ng/ml to about 50-100 μg/ml. Pharmaceutical dosage unit forms are prepared to provide from about 20 mg to about 300 mg and from about 25 to about 200 mg, or from about 25 up to about 100 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the compositions provided herein.

Thus, effective concentrations or amount of the endothelin antagonist, such as sitaxsentan or sitaxsentan, sodium is mixed with a suitable pharmaceutical carrier or vehicle for systemic, topical or local administration to form the pharmaceutical composition. The endothelin antagonist, such as sitaxsentan or sitaxsentan, sodium is included in an amount effective for treating or preventing sleep apnea.

The compositions are intended to be administered by a suitable route, including orally, parenterally, rectally, topically and locally. The endothelin antagonist, such as sitaxsentan or sitaxsentan, sodium is formulated and administered in unit-dosage forms such as tablets, capsules, pills, powders, granules, sterile parenteral solutions or suspensions, and oral solutions or suspensions, and oil-water emulsions containing suitable quantities of the active ingredient or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Lactose-free compositions provided herein can contain excipients that are well known in the art and are listed, for example, in the US. Pharmacopeia (USP) 25-NF20 (2002). In general, lactose-free compositions contains active ingredients, a binder/filler, and a lubricant in pharmaceutically compatible and pharmaceutically acceptable amounts. Particular lactose-free dosage forms contain active ingredients, microcrystalline cellulose, pre-gelatinized starch, and magnesium stearate.

Further provided are anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

a. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art. Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In certain embodiments, the formulations are solid dosage forms, such as capsules or tablets. The tablets, pills, capsules, troches and the like can contain any of the following ingredients, or conjugates of a similar nature: a binder; a filler, a diluent; a disintegrating agent; a lubricant; a glidant; a sweetening agent; and a flavoring agent. Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103, AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103 and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions herein is present in from about 50 to about 99 weight percent of the pharmaceutical composition or dosage form.

Disintegrants are used in the compositions provided herein to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms provided herein. The amount of disintegrant used varies based upon the type of formulation, and is readily discernible to those of ordinary skill in the art. Typical pharmaceutical compositions contain from about 0.5 to about 15 weight percent of disintegrant, or from about 1 to about 5 weight percent of disintegrant.

Disintegrants that can be used in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL®200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are used in an amount of less than about 1 weight percent of the pharmaceutical compositions or dosage forms into which they are incorporated.

If oral administration is desired, the endothelin antagonist, such as sitaxsentan or sitaxsentan, sodium could be provided in a composition that is formulated as enteric coating tablets, sugar-coated tablets, film-coated tablets or multiple compressed tablets. Enteric coating tablets protect the active ingredient from the acidic environment of the stomach. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges. The composition may also be formulated in combination with an antacid or other such ingredient.

When the dosage unit form is a capsule, it can contain, in addition to material of the above type, a liquid carrier such as a fatty oil. In a gelatin capsule, the solution or suspension containing the endothelin antagonist, such as sitaxsentan or sitaxsentan, sodium, in for example propylene carbonate, vegetable oils or triglycerides, is encapsulated in the capsule. Such solutions, and the preparation and encapsulation thereof, are disclosed in U.S. Pat. Nos. 4,328,245; 4,409,239; and 4,410,545.

The active ingredient can also be mixed with other active materials which do not impair the desired action, or with materials that supplement the desired action, such as antacids, H2 blockers, and diuretics. Higher concentrations, up to about 98% by weight of the active ingredient may be included.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative.

An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia.

Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

The pharmaceutical compositions containing active ingredients in micellar form can be prepared as described in U.S. Pat. No. 6,350,458. Such pharmaceutical compositions are particularly effective in oral, nasal and buccal applications.

In certain embodiments, formulations include, but are not limited to, those containing sitaxsentan or a pharmaceutically acceptable salt thereof, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In certain embodiments, sitaxsentan or a pharmaceutically acceptable salt thereof is formulated as an oral tablet containing about 50 mg, about 75 mg, about 100 mg, about 150 mg, about 200 mg, about 250 mg, about 300 mg, about 350 mg of the active ingredient. The capsule can contain inactive ingredients, such as polyethylene glycol 400, polysorbate 20, povidone, and butylated hydroxyanisole. The capsule shell can contain gelatin, sorbitol special glycerin blend and titanium dioxide.
Exemplary Oral Tablet Formulations

In certain embodiments, the methods provided herein encompass administration of oral tablets containing sitaxsentan sodium. In one embodiment, the oral tablet further contains a buffer. In one embodiment, the oral tablet further contains an antioxidant. In one embodiment, the oral tablet further contains a moisture barrier coating.

In some embodiments, the tablets contain excipients, including, but not limited to an antioxidant, such as sodium ascorbate, glycine, sodium metabisulfite, ascorbyl palmitate, disodium edetate (EDTA) or a combination thereof; a binding agent, such as hydroxypropyl methylcellulose; a diluent, such as lactose monohydrate, including lactose monohydrate fast flo (intragranular) and lactose monohydrate fast flo (extragranular) and microcrystalline cellulose and a buffer, such as phosphate buffer. The tablet can further contain one or more excipients selected from a lubricant, a disintegrant and a bulking agent.

In certain embodiments, the amount of sitaxsentan sodium in the oral tablet is from about 5% to about 40% of the total weight of the composition. In certain embodiments, the amount of sitaxsentan sodium is from about 7% to about 35%, 10% to about 30%, 12% to about 32%, 15% to about 30%, 17% to about 27%, 15% to about 25% of the total weight of the composition. In certain embodiments, the amount of sitaxsentan sodium is about 5%, 7%, 9%, 10%, 12%, 15%, 17%, 20%, 22%, 25%, 27%, 30%, 35% or 40% of the total weight of the composition. In certain embodiments, the amount of sitaxsentan sodium is about 20%.

In certain embodiments, the oral tablet contains about 10 mg, 20 mg, 25 mg, 30 mg, 40 mg, 50 mg, 60 mg, 70 mg, 80 mg, 90 mg, 100 mg, 125 mg, 150 mg, 175 mg, 200 mg, 225 mg, 250 mg, 275 mg, 280 mg, 300 mg or 350 mg of sitaxsentan sodium.

In certain embodiments, the tablets contain a combination of two antioxidants, such as ascorbyl palmitate and EDTA, disodium. In certain embodiments, the amount of ascorbyl palmitate in the formulation is in a range from about 0.05% to about 3% of the total weight of the tablet. In other embodiments, the amount of ascorbyl palmitate is in a range from about 0.07% to about 1.5%, 0.1% to about 1%, 0.15% to about 0.5% of the total weight of the tablet. In certain embodiments, the amount of ascorbyl palmitate in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1%. In certain embodiments, the amount of ascorbyl palmitate in the formulation is about 0.2% of the total weight of the tablet.

In certain embodiments, the amount of ascorbyl palmitate in the oral tablet is from about 0.1 mg to about 5 mg, about 0.5 mg to about 4 mg, about 0.7 mg to about 3 mg or about 1 mg to about 2 mg. In certain embodiments, the amount of ascorbyl palmitate in the oral tablet is about 0.1 mg, 0.5 mg, 0.7 mg, 1 mg, 1.3 mg, 1.5 mg, 1.7 mg, 2 mg, 2.5 mg or about 3 mg. In certain embodiments, the amount of ascorbyl palmitate in the formulation is about 1 mg.

In certain embodiments, the amount of EDTA, disodium in the formulation is in a range from about 0.05% to about 3% by weight of the total weight of the tablet. In other embodiments, the amount of EDTA, disodium is in a range from about 0.07% to about 1.5%, 0.1% to about 1%, 0.15% to about 0.5% of the total weight of the tablet. In certain embodiments, the amount of EDTA, disodium in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1%. In certain embodiments, the amount of EDTA, disodium in the formulation is about 0.2% of the total weight of the tablet.

In certain embodiments, the amount of EDTA, disodium in the oral tablet is from about 0.1 mg to about 5 mg, about 0.5 mg to about 4 mg, about 0.7 mg to about 3 mg or about 1 mg to about 2 mg. In certain embodiments, the amount of EDTA, disodium in the oral tablet is about 0.1 mg, 0.5 mg, 0.7 mg, 1 mg, 1.3 mg, 1.5 mg, 1.7 mg, 2 mg, 2.5 mg or about 3 mg. In certain embodiments, the amount of EDTA, disodium in the oral tablet is about 1 mg.

In certain embodiments, the tablets contain a combination of diluents, such as microcrystalline cellulose (AVICEL PH 102), lactose monohydrate fast flo (intragranular) and lactose monohydrate fast flo (extragranular). In certain embodiments, the amount of lactose monohydrate fast flo (intragranular) in the oral tablet is from about 5% to about 30% of the total weight of the composition. In certain embodiments, the amount of lactose monohydrate fast flo (intragranular) is from about 7% to about 25%, from about 10% to about 20%, from about 13% to about 20% of the total weight of the tablet. In certain embodiments, the amount of lactose monohydrate fast flo (intragranular) is about 5%, 7%, 10%, 13%, 14%, 15%, 15.5%, 16%, 16.1%, 16.2%, 16.3%, 16.4%, 16.5%, 16.6%, 16.7%, 16.8%, 16.9%, 17%, 17.5%, 18%, 18.5%, 19%, 20%, 25% or 30% of the total weight of the tablet. In certain embodiments, the amount of lactose monohydrate fast flo (intragranular) is about 16.9% of the total weight of the tablet.

In certain embodiments, the amount of lactose monohydrate fast flo (intragranular) is from about 40 mg to about 100 mg, from about 45 mg to about 95 mg, from about 50 mg to about 90 mg. In certain embodiments, the amount of lactose monohydrate fast flo (intragranular) is about 40 mg, 45 mg, 50 mg, 55 mg, 60 mg, 65 mg, 70 mg, 75 mg, 80 mg, 81 mg, 82 mg, 83 mg, 83.5 mg, 84 mg, 84.1 mg, 84.2 mg, 84.3 mg, 84.4 mg, 84.5 mg, 84.6 mg, 84.7 mg, 85 mg, 85.5 mg, 90 mg, 90.5 mg or 100 mg. In certain embodiments, the amount of lactose monohydrate fast flo (intragranular) is about 84.3 mg.

In certain embodiments, the amount of lactose monohydrate fast flo (extragranular) is from about 7% to about 25%, from about 10% to about 20%, from about 13% to about 20% of the total weight of the tablet. In certain embodiments, the amount of lactose monohydrate fast flo (extragranular) is about 5%, 7%, 10%, 13%, 14%, 15%, 15.5%, 16%, 16.1%, 16.2%, 16.3%, 16.4%, 16.5%, 16.6%, 16.7%, 16.8%, 16.9%, 17%, 17.5%, 18%, 18.5%, 19%, 20%, 25% or 30% of the total weight of the tablet. In certain embodiments, the amount of lactose monohydrate fast flo (extragranular) is about 16.4% of the total weight of the tablet. In certain embodiments, the amount of lactose monohydrate fast flo (extragranular) in the oral tablet is from about 40 mg to about 100 mg, from about 45 mg to about 95 mg, from about 50 mg to about 90 mg. In certain embodiments, the amount of lactose monohydrate fast flo (extragranular) is about 40 mg, 45 mg, 50 mg, 55 mg, 60 mg, 65 mg, 70 mg, 75 mg, 80 mg, 81 mg, 81.3 mg, 81.5 mg, 81.8 mg, 82 mg, 82.3 mg, 82.5 mg, 82.7 mg, 83 mg, 83.5 mg, 84 mg, 85 mg, 85.5 mg, 90 mg, 90.5 mg or 100 mg. In certain embodiments, the amount of lactose monohydrate fast flo (intragranular) is about 82 mg.

In certain embodiments, the amount of microcrystalline cellulose (Avicel PH 102) in the oral tablet is from about 10% to about 50% of the total weight of the composition. In certain embodiments, the amount of microcrystalline cellulose (Avicel PH 102) is from about 15% to about 45%, from about 20% to about 43%, from about 25% to about 40% of the total weight of the tablet. In certain embodiments, the amount of microcrystalline cellulose (Avicel PH 102) is about 15%, 17%, 20%, 23%, 25%, 27%, 30%, 32%, 34%, 35%, 37%, 40%, 42%, 45% or 50% of the total weight of the tablet. In certain embodiments, the amount of microcrystalline cellulose (Avicel PH 102) is about 35% of the total weight of the tablet.

In certain embodiments, the amount of microcrystalline cellulose (Avicel PH 102) in the oral tablet is from about 130 mg to about 300 mg. In certain embodiments, the amount of microcrystalline cellulose (Avicel PH 102) is from about 140 mg to about 275 mg or about 150 mg to about 250 mg. In certain embodiments, the amount of microcrystalline cellulose (Avicel PH 102) is about 150 mg, 160 mg, 165 mg, 170 mg, 175 mg, 180 mg, 185 mg, 190 mg or 200 mg. In certain embodiments, the amount of microcrystalline cellulose (Avicel PH 102) in the oral tablet is about 175 mg.

In certain embodiments, the binding agent is hydroxypropyl methylcellulose (E-5P). In certain embodiments, the amount of hydroxypropyl methylcellulose (E-5P) in the tablet is from about 0.5% to about 20% of the total weight of the composition. In certain embodiments, the amount of hydroxypropyl methylcellulose (E-5P) is from about 1% to about 15%, from about 2% to about 10%, from about 3% to about 8% of the total weight of the tablet. In certain embodiments, the amount of hydroxypropyl methylcellulose (E-5P) is about 1%, 2%, 3%, 4%, 5%, 6%, 7%, 8%, 9% or 10% of the total weight of the tablet. In certain embodiments, the amount of hydroxypropyl methylcellulose (E-5P) is about 5% of the total weight of the tablet.

In certain embodiments, the amount of hydroxypropyl methylcellulose (E-5P) in the tablet is from about 5 mg to about 50 mg, about 10 mg to about 40 mg or about 15 mg to about 30 mg. In certain embodiments, the amount of hydroxypropyl methylcellulose (E-5P) in the tablet is about 10 mg, 15 mg, 20 mg, 22 mg, 25 mg, 27 mg, 30 mg, 35 mg or about 40 mg. In certain embodiments, the amount of hydroxypropyl methylcellulose (E-5P) in the tablet is about 25 mg.

The formulations of sitaxsentan sodium provided herein are stable at neutral pH. In certain embodiments, buffer agent mixture, such as sodium phosphate monobasic monohydrate and sodium phosphate dibasic anhydrous is used to improve drug stability in the tablets. In certain embodiments, the amount of sodium phosphate, monobasic monohydrate ranges from about 0.05% to about 3% by weight of the total weight of the tablet. In other embodiments, the amount of sodium phosphate, monobasic monohydrate is in a range from about 0.07% to about 1.5%, 0.1% to about 1%, 0.15% to about 0.5% of the total weight of the tablet. In certain embodiments, the amount of sodium phosphate, monobasic monohydrate in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1. %. In certain embodiments, the amount of sodium phosphate, monobasic monohydrate in the formulation is about 0.1% of the total weight of the tablet.

In certain embodiments, the amount of sodium phosphate, monobasic monohydrate in the oral tablet is from about 0.1 mg to about 3 mg, about 0.2 mg to about 2.5 mg, about 0.5 mg to about 2 mg or about 0.6 mg to about 1 mg. In certain embodiments, the amount of sodium phosphate, monobasic monohydrate in the oral tablet is about 0.1 mg, 0.2 mg, 0.3 mg, 0.4 mg, 0.5 mg, 0.6 mg, 0.7 mg, 0.8 mg, 0.9 mg or about 1 mg. In certain embodiments, the amount of sodium phosphate, monobasic monohydrate in the oral tablet is about 0.6 mg.

In certain embodiments, the amount of sodium phosphate, dibasic anhydrous ranges from about 0.05% to about 3% by weight of the total weight of the tablet. In other embodiments, the amount of sodium phosphate dibasic is in a range from about 0.07% to about 1.5%, 0.1% to about 1%, 0.15% to about 0.5% of the total weight of the tablet. In certain embodiments, the amount of sodium phosphate dibasic in the formulation is about 0.05%, 0.07%, 0.09%, 0.1%, 0.12%, 0.15%, 0.17%, 0.18%, 0.2%, 0.23%, 0.25%, 0.27%, 0.3%, 0.35%, 0.4%, 0.45%, 0.5%, 0.7% or 1. %. In certain embodiments, the amount of sodium phosphate dibasic in the formulation is about 0.2% of the total weight of the tablet.

In certain embodiments, the amount of sodium phosphate, dibasic anhydrous in the oral tablet is from about 0.1 mg to about 3.5 mg, about 0.5 mg to about 2.5 mg, or about 0.7 mg to about 2 mg. In certain embodiments, the amount of sodium phosphate, dibasic anhydrous in the oral tablet is about 0.1 mg, 0.3 mg, 0.5 mg, 0.7 mg, 0.9 mg, 1 mg, 1.1 mg, 1.3 mg, 1.5 mg, 1.7 mg or 2 mg. In certain embodiments, the amount of sodium phosphate, dibasic anhydrous in the oral tablet is about 1.1 mg.

In certain embodiments, the tablet contains disintegrants, such as Sodium Starch Glycoloate (intragranular) and Sodium Starch Glycoloate (extragranular). In certain embodiments, the amount of Sodium Starch Glycoloate (intragranular) in the tablet is from about 0.1% to about 10% of the total weight of the composition. In certain embodiments, the amount of Sodium Starch Glycoloate (intragranular) is from about 0.5% to about 8%, from about 1% to about 5%, from about 2% to about 4% of the total weight of the tablet. In certain embodiments, the amount of Sodium Starch Glycoloate (intragranular) is about 0.5%, 1%, 1.5%, 1.7%, 2%, 2.3%, 2.5%, 2.7%, 3%, 3.5%, 4% or 5% of the total weight of the tablet. In certain embodiments, the amount of Sodium Starch Glycoloate (intragranular) is about 2.5% of the total weight of the tablet. In certain embodiments, the amount of Sodium Starch Glycoloate (intragranular) is from about 30 mg to about 5 mg, from about 20 mg to about 10 mg, from about 15 to about 10 mg. In certain embodiments, the amount of Sodium Starch Glycoloate (intragranular) is about 5 mg, 7 mg, 10 mg, 11 mg, 11.5 mg, 12 mg, 12.5 mg, 13 mg, 15 mg or 20 mg. In certain embodiments, the amount of Sodium Starch Glycoloate (intragranular) is about 12.5 mg.

In certain embodiments, the amount of Sodium Starch Glycoloate (extragranular) in the tablet is from about 0.1% to about 10% of the total weight of the composition. In certain embodiments, the amount of Sodium Starch Glycoloate (extragranular) is from about 0.5% to about 8%, from about 1% to about 5%, from about 2% to about 4% of the total weight of the tablet. In certain embodiments, the amount of Sodium Starch Glycoloate (extragranular) is about 0.5%, 1%, 1.5%, 1.7%, 2%, 2.3%, 2.5%, 2.7%, 3%, 3.5%, 4% or 5% of the total weight of the tablet. In certain embodiments, the amount of Sodium Starch Glycoloate (extragranular) is about 2.5% of the total weight of the tablet. In certain embodiments, the amount of Sodium Starch Glycoloate (extragranular) is from about 30 mg to about 5 mg, from about 20 mg to about 10 mg, from about 15 to about 10 mg. In certain embodiments, the amount of Sodium Starch Glycoloate (extragranular) is about 5 mg, 7 mg, 10 mg, 11 mg, 11.5 mg, 12 mg, 12.5 mg, 13 mg, 15 mg or 20 mg. In certain embodiments, the amount of Sodium Starch Glycoloate (extragranular) is about 12.5 mg.

In certain embodiments, the tablet contains a lubricant, such as magnesium stearate. In certain embodiments, the amount of magnesium stearate in the tablet is from about 0.1% to about 8% of the total weight of the composition. In certain embodiments, the amount of magnesium stearate is from about 0.5% to about 6%, from about 0.7% to about 5%, from about 1% to about 4% of the total weight of the tablet. In certain embodiments, the amount of magnesium stearate is about 0.5%, 0.7%, 1%, 1.2%, 1.5%, 1.7%, 2%, 2.5% or 3% of the total weight of the tablet. In certain embodiments, the amount of magnesium stearate is about 2.5% of the total weight of the tablet. In certain embodiments, the amount of magnesium stearate in the tablet is from about 15 mg to about 1 mg. In certain embodiments, the amount of magnesium stearate is from about 10 mg to about 3 mg or from about 7 mg to about 5 mg. In certain embodiments, the amount of magnesium stearate is about 3 mg, 4 mg, 4.5 mg, 5 mg, 6 mg, 7 mg, 8 mg, 9 mg or 10 mg. In certain embodiments, the amount of magnesium stearate is about 5 mg.

The tablet formulations provided herein contain a moisture barrier coating. Suitable coating materials are known in the art and include, but are not limited to coating agents either of cellulose origin such as cellulose phthalate (Sepifilm, Pharmacoat), or of polyvinyl origin of Sepifilm ECL type, or of saccharose origin such as the sugar for sugar-coating of Sepisperse DR, AS, AP OR K (coloured) type, such as Sepisperse Dry 3202 Yellow, Blue Opadry, Eudragit EPO and Opadry AMB. The coating serves as a moisture barrier to hinder oxidation of sitaxsentan sodium. In certain embodiments, the coating materials are Sepifilm LP014/Sepisperse Dry 3202 Yellow (Sepifilm/Sepisperse) (3/2 wt/wt) at from about 1 to about 7% or about 4% tablet weight gain. In certain embodiments, the coating material is Sepifilm LP014/Sepisperse Dry 3202 Yellow (Sepifilm/Sepisperse). In certain embodiments, the Sepifilm/Sepisperse ratio is 1:2, 1:1 or 3:2 wt/wt. In certain embodiments, the Sepifilm/Sepisperse coating is at about 1%, 2%, 3%, 4%, 5%, 6% or 7% tablet weight gain. In certain embodiments, the Sepifilm/Sepisperse coating is at about 1.6% tablet weight gain. In certain embodiments, the Sepisperse Dry 3202 (yellow) is at about 0.5%, 0.8%, 1%, 1.3%, 1.6%, 2%, 2.4%, 2.5%, 3% or 4% tablet weight gain. In certain embodiments, the Sepisperse Dry 3202 (yellow) is at about 2.4% tablet weight gain. In certain embodiments, the Sepisperse Dry 3202 (yellow) is at about 1 mg, 3 mg, 5 mg, 6 mg, 7 mg, 8 mg, 9 mg, 10 mg, 13 mg 15 mg or 20 mg per tablet. In certain embodiments, the Sepisperse Dry 3202 (yellow) is at about 8 mg per tablet. In certain embodiments, the Sepifilm LP 014 is at about 0.5%, 1%, 1.5%, 2%, 2.2%, 2.4%, 2.6%, 3%, 3.5% or 4% tablet weight gain. In certain embodiments, the Sepifilm LP 014 is at about 2.4% tablet weight gain. In certain embodiments, the Sepifilm LP 014 is at about 5 mg, 7 mg, 9 mg, 10 mg, 11 mg, 12 mg, 13 mg, 15 mg, 17 mg or 20 mg per tablet. In certain embodiments, the Sepifilm LP 014 coating is at about 12 mg per tablet.

In certain embodiments, the tablet contains sitaxsentan sodium, microcrystalline cellulose, lactose monohydrate fast flo (intragranular), lactose monohydrate fast flo (extragranular), hydroxypropyl methylcellulose E-5P, ascorbyl palmitate, disodium EDTA, sodium phosphate monobasic, monohydrate, sodium phosphate dibasic, anhydrous, Sodium Starch Glycoloate (intragranular), Sodium Starch Glycoloate (extragranular), magnesium stearate and a coating of Sepifilm LP014/Sepisperse Dry 3202 Yellow.

In certain embodiments, the tablet contains about 20% sitaxsentan sodium, about 35% microcrystalline cellulose, about 16.9% lactose monohydrate fast flo (intragranular), about 16.4% lactose monohydrate fast flo (extragranular), about 5.0% hydroxypropyl methylcellulose E-5P, about 0.2% ascorbyl palmitate, about 0.2% disodium (EDTA), about 0.1% sodium phosphate monobasic, monohydrate, about 0.2% sodium phosphate dibasic, anhydrous, about 2.5% Sodium Starch Glycoloate (extragranular), about 2.5% Sodium Starch Glycoloate (intragranular) and about 1% magnesium stearate. The tablet further contains a coating of Sepifilm LP014 at about 2.4% weight gain and Sepisperse Dry 3202 Yellow at about 1.6% weight gain.

In certain embodiments, the oral tablet provided herein is a 500 mg tablet that contains about 100 mg sitaxsentan sodium, about 1.0 mg ascorbyl palmitate, about 1.0 mg disodium edetate (EDTA), about 25 mg hydroxypropyl methylcellulose E-5P, about 84.3 lactose monohydrate fast flo (intragranular), about 82 mg lactose monohydrate fast flo (extragranular), about 175 mg microcrystalline cellulose, about 0.6 mg sodium phosphate monobasic, monohydrate, about 1.1 mg sodium phosphate dibasic, anhydrous, about 12.5 mg Sodium Starch Glycoloate (extragranular), about 12.5 mg Sodium Starch Glycoloate (intragranular), about 5 mg magnesium stearate, non-bovine and about 192.5 mg purified water. The tablet further contains a coating of Sepifilm LP014 at about 12 mg and Sepisperse Dry 3202 Yellow at about 8 mg.

b. Sustained Release Dosage Form

Active ingredients provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In one embodiment, a pump may be used (see, Sefton, CRC Crit. Ref Biomed. Eng. 14:201 (1987); Buchwald et al., Surgery 88:507 (1980); Saudek et al., N. Engl. J. Med. 321:574 (1989). In another embodiment, polymeric materials can be used.

In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

c. Parenteral Administration

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of sitaxsentan or a pharmaceutically acceptable salt thereof is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active ingredient is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, or more than 1% w/w of sitaxsentan to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

Sitaxsentan or a pharmaceutically acceptable salt thereof may be suspended in micronized or other suitable form or may be derivatized to produce a more soluble active product or to produce a prodrug. The form of the resulting mixture depends upon a number of factors, including the intended mode of administration and the solubility of sitaxsentan or a pharmaceutically acceptable salt thereof in the selected carrier or vehicle. The effective concentration is sufficient for ameliorating the symptoms of the condition and may be empirically determined.

d. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving the active ingredient, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-350 mg, or 100-300 mg) or multiple dosages of sitaxsentan or a pharmaceutically acceptable salt thereof. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, 5-35 mg, or about 9-30 mg of lyophilized powder, is added per mL of sterile water or other suitable carrier. The precise amount can be empirically determined.
Exemplary Lyophilized Formulations

In certain embodiments, provided herein are stable lyophilized powders of sitaxsentan sodium. The lyophilized powder contains an antioxidant, a buffer and a bulking agent. In the lyophilized powders provided herein, the amount of sitaxsentan sodium present is in a range from about 25% to about 60% by total weight of the lyophilized powder. In certain embodiments, the amount of sitaxsentan sodium is from about 30% to about 50% or about 35% to about 45% by total weight of the lyophilized powder. In certain embodiments, the amount of sitaxsentan sodium is about 30%, 33%, 35%, 37%, 40%, 41%, 43%, 45%, 47%, 50%, 53%, 55% or 60% by total weight of the lyophilized powder. In one embodiment, the amount of sitaxsentan sodium in the lyophilized powder is about 41% by total weight of the lyophilized powder.

In certain embodiments, the lyophilized powder contains an antioxidant, such as sodium sulfite, sodium bisulfite, sodium metasulfite, monothioglycerol, ascorbic acid or a combination thereof. In one embodiment, the antioxidant is monothioglycerol. In one embodiment, the antioxidant is a combination of ascorbic acid, sodium sulfite and sodium bisulfite. In certain embodiments, the lyophilized formulations provided herein have improved stability upon reconstitution as compared to the known lyophilized formulations of sitaxsentan sodium (see WO 98/49162).

In certain embodiments, the antioxidant is monothioglycerol. In certain embodiments, the monothioglycerol is present in an amount ranging from about 10% to about 30% by total weight of the lyophilized powder. In certain embodiments, the monothioglycerol is present in an amount ranging from about 12% to about 25% or about 15% to about 20% by total weight of the lyophilized powder. In certain embodiments, the amount of monothioglycerol in the lyophilized powder is about 10%, 12%, 14%, 15%, 15.5%, 16%, 16.2%, 16.4%, 16.8%, 17%, 17.5%, 19%, 22%, 25% or 30% by total weight of the lyophilized powder. In certain embodiments, the amount of monothioglycerol is about 16.4% by total weight of the lyophilized powder.

In certain embodiments, the sodium sulfite is present in an amount from about 1% to about 6% by total weight of the lyophilized powder. In other embodiments, the sodium sulfite is present in an amount from about 1.5% to about 5% or about 2% to about 4%. In certain embodiments, the amount of sodium sulfite is about 1%, 1.5%, 2%, 2.5%, 3%, 3.3%, 3.5%, 3.8%, 4%, 4.5% or 5% by total weight of the lyophilized powder. In one embodiment, the amount of sodium sulfite is about 3.3% by total weight of the lyophilized powder.

In certain embodiments, the ascorbic acid is present in an amount from about 1% to about 6% by total weight of the lyophilized powder. In other embodiments, the ascorbic acid is present in an amount from about 1.5% to about 5% or about 2% to about 4%. In certain embodiments, the amount of ascorbic acid is about 1%, 1.5%, 2%, 2.5%, 3%, 3.3%, 3.5%, 3.8%, 4%, 4.5% or 5% by total weight of the lyophilized powder. In one embodiment, the amount of ascorbic acid is about 3.3% by total weight of the lyophilized powder.

In certain embodiments, the sodium bisulfite is present in an amount from about 5% to about 15% or about 8% to about 12% by total weight of the lyophilized powder. In certain embodiments, the sodium bisulfite is present in an amount from about 5%, 6%, 7%, 8%, 9%, 10%, 10.3%, 10.5%, 10.8%, 11%, 11.5%, 12% or 15% by total weight of the lyophilized powder. In one embodiment, the amount of sodium bisulfite is about 10.8% by total weight of the lyophilized powder.

In one embodiment, the antioxidant is a combination of ascorbic acid, sodium sulfite and sodium bisulfite. In one embodiment, the amount of ascorbic acid in the lyophilized powder is about 3.3%, the amount of sodium sulfite is about 3.3% and the amount of sodium bisulfite is about 10.8% by total weight of the lyophilized powder.

In one embodiment, the lyophilized powder also contains one or more of the following excipients: a buffer, such as sodium or potassium phosphate, or citrate; and a bulking agent, such as glucose, dextrose, maltose, sucrose, lactose, sorbitol, mannitol, glycine, polyvinylpyrrolidone, dextran. In one embodiment, the bulking agent is selected from dextrose, D-mannitol or sorbitol.

In certain embodiments, the lyophilized powders provided herein contain a phosphate buffer. In certain embodiments, the phosphate buffer is present in a concentration of about 10 mM, about 15 mM, about 20 mM, about 25 mM or about 30 mM. In certain embodiments, the phosphate buffer is present in a concentration of 20 mM. In certain embodiments, the phosphate buffer is present in a concentration of 20 mM, and the constituted formulation has a pH of about 7.

In certain embodiments, the lyophilized powders provided herein contain a citrate buffer. In one embodiment, the citrate buffer is sodium citrate dihydrate. In certain embodiments, the amount of sodium citrate dihydrate is from about 5% to about 15%, about 6% to about 12% or about 7% to about 10% by total weight of the lyophilized powder. In certain embodiments, the amount of sodium citrate dihydrate in the lyophilized powder is about 5%, 6%, 7%, 7.5%, 8%, 8.3%, 8.5%, 8.8%, 9%, 9.5%, 10%, 12% or about 15% by total weight of the lyophilized powder. In certain embodiments, the constituted formulation has a pH of about 5 to 10, or about 6.

In certain embodiments, the lyophilized powder provided herein contains dextrose in an amount ranging from about 30% to about 60% by total weight of the lyophilized powder. In certain embodiments, the amount of dextrose is about 30%, 35%, 40%, 45%, 50% or 60% by total weight of the lyophilized powder. In certain embodiments, the amount of dextrose is about 40% by total weight of the lyophilized powder. In certain embodiments, the lyophilized powder provided herein contains mannitol in an amount ranging from about 20% to about 50% by total weight of the lyophilized powder. In certain embodiments, the amount of mannitol is about 20%, 25%, 30%, 32%, 32.5%, 32.8%, 33%, 34%, 37%, 40%, 45% or 50% by total weight of the lyophilized powder. In certain embodiments, the amount of mannitol is about 32.8% by total weight of the lyophilized powder.

In certain embodiments, the lyophilized powder provided herein contains about 41% of sitaxsentan sodium, about 3.3% ascorbic acid, about 3.3% sodium sulfite and about 10.8% mg sodium bisulfite, about 8.8% sodium citrate dihydrate and about 32.8% mannitol by total weight of the lyophilized powder. In certain embodiments, the lyophilized powder has the following composition:

Sitaxsentan Sodium Lyophilized Formulation

| | Quantity in a 10 mL vial |
|---|---|
| Component | (mg/vial) |
| Sitaxsentan Sodium | 250.0 |
| Sodium Citrate Dihydrate | 53.5 |
| L-Ascorbic Acid | 20.0 |
| D-Mannitol | 200.0 |
| Sodium Bisulfite | 66.0 |
| Sodium Sulfite | 20.0 |
| Sodium Hydroxide or Hydrochloride Acid | QS to pH 6 |

In certain embodiments, the lyophilized powder provided herein contains about 40 to about 30% of sitaxsentan sodium, about 4 to about 6% ascorbic acid, about 6 to about 8% sodium citrate dihydrate, about 50 to about 60% D-mannitol and about 1 to about 2% citric acid monohydrate by total weight of the lyophilized powder. In certain embodiments, the lyophilized powder provided herein contains about 33% of sitaxsentan sodium, about 5.3% ascorbic acid, about 7.6% sodium citrate dihydrate, about 53% D-mannitol and 0.13% citric acid monohydrate by total weight of the lyophilized powder. In one embodiment, the lyophilized powder has the following composition:

Sitaxsentan Sodium Lyophilized Formulation

| | Quantity in a 10 mL vial |
|---|---|
| Component | (mg/vial) |
| Sitaxsentan Sodium | 250.0 |
| Sodium Citrate Dihydrate | 57.1 |
| L-Ascorbic Acid | 40.0 |
| D-Mannitol | 400.0 |
| Citric Acid Monohydrate | 1.3 |
| Sodium Hydroxide or Hydrochloride Acid | QS to pH 6.8 |

In certain embodiments, the lyophilized powder provided herein contains about 40 to about 30% of sitaxsentan sodium, about 4 to about 6% ascorbic acid, about 3 to about 4% sodium phosphate dibasic heptahydrate, about 50 to about 60% D-mannitol and about 1.5 to about 2.5% sodium phosphate monobasic monohydrate by total weight of the lyophilized powder. In certain embodiments, the lyophilized powder provided herein contains about 34% of sitaxsentan sodium, about 5.5% ascorbic acid, about 3.7% sodium phosphate dibasic heptahydrate, about 55% D-mannitol and 1.9% sodium phosphate monobasic monohydrate by total weight of the lyophilized powder. In one embodiment, the lyophilized powder has the following composition:

Sitaxsentan Sodium Lyophilized Formulation

| | Quantity in a 10 mL vial |
|---|---|
| Component | (mg/vial) |
| Sitaxsentan Sodium | 250.0 |
| Sodium Phosphate Dibasic Heptahydrate | 26.8 |
| L-Ascorbic Acid | 40.0 |
| D-Mannitol | 400.0 |
| Sodium Phosphate Monobasic | 13.9 |
| Monohydrate | |
| Sodium Hydroxide or Hydrochloride Acid | QS to pH 6.8 |

The lyophilized formulations of sitaxsentan sodium provided herein can be administered to a patient in need thereof using standard therapeutic methods for delivering sitaxsentan sodium including, but not limited to, the methods described herein. In one embodiment, the lyophilized sitaxsentan sodium is administered by dissolving a therapeutically effective amount of the lyophilized sitaxsentan sodium provided herein in a pharmaceutically acceptable solvent to produce a pharmaceutically acceptable solution, and administering the solution (such as by intravenous injection) to the patient.

The lyophilized sitaxsentan sodium formulation provided herein can be constituted for parenteral administration to a patient using any pharmaceutically acceptable diluent. Such diluents include, but are not limited to Sterile Water for Injection, USP, Sterile Bacteriostatic Water for Injection, saline, USP (benzyl alcohol or parabens preserved). Any quantity of diluent may be used to constitute the lyophilized sitaxsentan sodium formulation such that a suitable solution for injection is prepared. Accordingly, the quantity of the diluent must be sufficient to dissolve the lyophilized sitaxsentan sodium. In one embodiment, 10-50 mL or 10 to 20 mL of a diluent are used to constitute the lyophilized sitaxsentan sodium formulation to yield a final concentration of, about 1-50 mg/mL, about 5-40 mg/mL, about 10-30 mg/mL or 10-25 mg/mL. In certain embodiments, the final concentration of sitaxsentan sodium in the reconstituted solution is about 25 mg/mL or about 12.5 mg/mL. The precise amount depends upon the indication treated. Such amount can be empirically determined. In some embodiments, the pH of the reconstituted solution is about 5 to about 10 or about 6 to about 8. In some embodiments, the pH of the reconstituted solution is about 5, 6, 7, 8, 9 or 10.

Constituted solutions of lyophilized sitaxsentan sodium can be administered to a patient promptly upon constitution. Alternatively, constituted solutions can be stored and used within about 1-72 hours, about 1-48 hours or about 1-24 hours. In some embodiments, the solution is used within 1 hour of preparation.

e. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The endothelin antagonist, such as sitaxsentan or sitaxsentan, sodium may be formulated for local or topical application, such as for topical application to the skin and mucous membranes, in the form of gels, creams, and lotions. Topical administration is contemplated for transdermal delivery and also for administration mucosa, or for inhalation therapies.

f. Compositions for Other Routes of Administration

Other routes of administration, such as topical application, transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

Dosages

In human therapeutics, the physician will determine the dosage regimen that is most appropriate according to a preventive or curative treatment and according to the age, weight, stage of the disease and other factors specific to the subject to be treated. In certain embodiments, dose rates of sitaxsentan sodium are from about 1 to about 350 mg per day for an adult, from about 1 to about 300 mg per day, from about 5 to about 250 mg per day, from about 5 to about 250 mg per day or from about 10 to 50 mg per day for an adult. Dose rates of from about 50 to about 300 mg per day are also contemplated herein. In certain embodiments, doses are about 5 mg, 10 mg, 15 mg, 20 mg, 25 mg, 30 mg, 35 mg, 40 mg, 45 mg, 50 mg, 60 mg, 70 mg, 80 mg, 100 mg, 125 mg, 150 mg, 175 mg or 200 mg per day per adult.

The amount of sitaxsentan sodium in the formulations provided herein which will be effective in the prevention or treatment of the symptoms of sleep apnea will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject.

Exemplary doses of a formulation include milligram or microgram amounts of the active compound per kilogram of subject or sample weight (e.g., from about 1 micrograms per kilogram to about 3 milligrams per kilogram, from about 10 micrograms per kilogram to about 3 milligrams per kilogram, from about 100 micrograms per kilogram to about 3 milligrams per kilogram, or from about 100 microgram per kilogram to about 2 milligrams per kilogram). In certain embodiments, the amount of sitaxsentan sodium administered is from about 0.01 to about 3 mg/kg for a subject in need thereof. In certain embodiments, the amount of sitaxsentan sodium administered is about 0.01, 0.05, 0.1, 0.2, 0.4, 0.8, 1.5, 2, 3 mg/kg of a subject. In the certain embodiments, the administration of sitaxsentan sodium is by intravenous injection.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

The amounts sufficient to prevent, manage, treat or ameliorate the symptoms of sleep apnea, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In another embodiment, the dosage of the formulation provided herein is administered to prevent, treat, manage, or ameliorate the symptoms of sleep apnea in a subject in a unit dose of from about 1 mg to 300 mg, 50 mg to 250 mg or 75 mg to 200 mg.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months, or 6 months.

Articles of Manufacture

The endothelin antagonist, such as sitaxsentan or sitaxsentan, sodiummay be packaged as articles of manufacture containing packaging material and a label that indicates that the endothelin antagonist, such as sitaxsentan or sitaxsentan, sodium is used for treating sleep apnea. The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,352. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the endothelin antagonist, such as sitaxsentan or sitaxsentan, sodium provided herein are contemplated herein.

Evaluation of the Activity

Standard physiological, pharmacological and biochemical procedures are available and are known to one of skill in the art to test the efficacy of the endothelin antagonist, such as sitaxsentan or sitaxsentan, sodium in the methods provided herein.

Combination Therapy

In the methods provided herein, the endothelin antagonist, such as sitaxsentan or sitaxsentan sodium may, for example, be employed alone, in combination with one or more other endothelin antagonists, or with another compound or therapies useful for the treatment of sleep apnea. For example, the formulations can be administered in combination with other compounds known to modulate the activity of endothelin receptor, such as the compounds described in U.S. Pat. Nos. 6,432,994; 6,683,103; 6,686,382; 6,248,767; 6,852,745; 5,783,705; 5,962,490; 5,594,021; 5,571,821; 5,591,761; 5,514,691. Several other endothelin antagonists are described in the literature as described above.

In some embodiments, the methods encompass administration of sitaxsentan sodium in combination with other therapies used in treatment of sleep apnea, such as behavioral changes, physical or mechanical therapy, such as oxygen administration, continuous positive airway pressure (CPAP), dental appliances or jaw adjustment devices, surgery or a combination thereof.

### EXAMPLES

### Example 1

### Preparation of 4-chloro-3-methyl-5-(2-(2-(6-methylbenzo[d][1,3]dioxol-5-yl) acetyl)-3-thienylsulfonamido)isoxazole, sodium salt or N-(4-chloro-3-methyl-5-isoxazolyl)-2-[2-methyl-4,5-(methylenedioxy)phenylacetyl]-thiophene-3-sulfonamide, sodium salt or N-(4-chloro-3-methyl-5-isoxazolyl)-2-[3,4-(methylenedioxy)-6-methylphenylacetyl]-thiophene-3-sulfonamide, sodium salt

### A. Preparation of (4-chloro-3-methyl-5-(2-(2-(6-methylbenzo[d][1,3]dioxol-5-yl)acetyl)-3-thienylsulfonamido)isoxazole

### 1. Preparation of 5-chloromethyl-6-methylbenzo[d][1,3]dioxole

To a mixture of methylene chloride (130 L), concentrated HCl (130 L), and tetrabuylammonium bromide (1.61 Kg) was added 5-methylbenzo[d][1,3]dioxole (10 Kg) followed by the slow addition of formaldehyde (14 L, 37 wt % in water). The mixture was stirred overnight. The organic layer was separated, dried with magnesium sulfate and concentrated to an oil. Hexane (180 L) was added and the mixture heated to boiling. The hot hexane solution was decanted from a heavy oily residue and evaporated to give almost pure 5-chloromethyl-6-methylbenzo[d][1,3]dioxole as a white solid. Recrystallization from hexane (50 L) gave 5-chloromethyl-6-methylbenzo[d][1,3]dioxole (80% recovery after recrystallization).

### 2. Formation of (4-chloro-3-methyl-5-(2-(2-(2-methylbenzod[d][1,3]dioxol-5-yl)acetyl)-3-thienylsulfonamido)isoxazole

A portion of a solution of 5-chloromethyl-6-methylbenzo[d][1,3]di-oxole (16.8 g, 0.09 mol) in tetrahydrofuran (THF)(120 mL) was added to a well stirred slurry of magnesium powder, (3.3 g, 0.136 g-atom, Alfa, or Johnson-Mathey, −20+100 mesh) in THF (120 mL) at room temperature. The resulting reaction admixture was warmed up to about 40-45° C. for about 2-3 min, causing the reaction to start. Once the magnesium was activated by the heating, and the reaction begun, the mixture was cooled and maintained at a temperature below about 8° C. The magnesium can be activated with dibromoethane in place of heat.

A flask containing the reaction mixture was cooled and the remaining solution of 5-chloromethlybenzo[d][1,3]dioxole added dropwise during 1.5 hours while maintaining an internal temperature below 8° C. Temperature control is important: if the Grignard is generated and kept below 8° C., no Wurtz coupling takes place. Longer times at higher temperatures promote the Wurtz coupling pathway. Wurtz coupling can be avoided by using high quality Mg and by keeping the temperature of the Grignard below about 8° C. and stirring vigorously. The reaction works fine at −20° C., so any temperature below 8° C. is acceptable at which the Grignard will form. The color of the reaction mixture turns greenish.

The reaction mixture was stirred for an additional 5 min at 0° C., while N²-methoxy-N²-methyl-3-(4-chloro-3-methyl-5-isoazolylsulfamoyl)-2-thio-phenecarboxamide (6.6 g, 0.018 mol) in anhydrous THF (90 mL) was charged into the addition funnel. The reaction mixture was degassed two times then the solution of N²-methoxy-N-methyl-3-(4-chloro-3-methyl-5-isoxazolylsulfamoyl)-2-thio-phenecarboxamide was added at 0° C. over 5 min. TLC of the reaction mixture (Silica, 12% MeOH/CH₂Cl₂) taken immediately after the addition shows no N²-methoxy-N-methyl-3-(4-chloro-3-methyl-5-isoxazolysulfamoyl)-2-thiophenecarboxamide.

The reaction mixture was transferred into a flask containing 1N HCl (400 mL, 0.4 mol HCl, ice-bath stirred), and the mixture stirred for 2 to 4 min, transferred into a separatory funnel and diluted with ethyl acetate (300 mL). The layers were separated after shaking. The water layer was extracted with additional ethyl acetate (150 mL) and the combined organics washed with half-brine. Following separation, THF was removed by drying the organic layer over sodium sulfate and concentrating under reduced pressure at about 39° C.

### B. Preparation of 4-chloro-3-methyl-5-(2-(2-(6-methylbenzo[d][1,3]dioxol-5yl)acetyl)-3-thienylsulfonamido)isoxazole, sodium salt

The product from part A was then re-dissolved in ethyl acetate and washed with saturated NaHCO₃ (5×50 mL) until the washings became colorless. The solution was washed with brine, dried over Na₂SO₄ and concentrated in vacuo to give a semicrystalline yellow residue. 100 mL of CH₂Cl₂ was added to the solution and the mixture stirred under nitrogen for from 5 to 10 minutes until a fine crystalline product was formed. Ether (150 mL) was added and the mixture stirred from an appropriate time (e.g., 10 min). The product was isolated by filtration, washed with a mixture of CH₂Cl₂/ether (1:2) (30 mL) then with ether (30 mL) and dried under reduced pressure. When prepared in accordance with the specific embodiments set forth above, the title product was produced in quantity of 7.3 g with a purity of around 85% (HPLC, RP, 40% acetonitrile/water, 0.1% TFA neutralized with ammonia to pH2.5, isocratic conditions, 1 mL/min).

The salt product from above was dissolved in water (600 mL) at 10° C., the solution stirred for a short period of time (e.g., 3 min) and then filtered through a layer of paper filters (e.g., 3 filters) with suction. In some cases, the large amount of impurities that are not soluble in water (10% or higher) slows down the filtration process extremely. This problem can be avoided by using a larger size filter during the filtration. Usually there is no problem with filtration if the purity of the crude salt is 90% or higher.

The greenish slightly turbid solution obtained from filtration was cooled in an ice bath and acidified to a pH of 2 using an acid such as 4N HCl. When the pH of the solution was 2, the product precipitates as a milky, non-filterable material. Slow dropwise addition of extra 4N HCl causes the product to form a fine, easily filterable precipitate. The pale yellow precipitate was filtered off, washed with water until neutral and pressed on the filter to get rid of excess of water). The obtained free acid was typically 95% pure as determined by HPLC.

The free acid form of the product was dissolved in ethyl acetate (about 100 mL), washed with brine (30 mL) to remove water. The dehydrated solution was shaken with cold saturated NaHCO₃ solution (2×30 mL), then with brine again, dried over Na₂SO₄ and concentrated in vacuo (bath temperature lower than 40° C.) to give a very bright yellow foam. After complete removal of the ethyl acetate from this product, CH₂Cl₂ (100 mL) was added and the mixture stirred for 5 to 10 min until the product became crystalline. Ether (150 mL) was added and stirring continued for 10 min longer. The formed solid was isolated by filtration, washed with a mixture of CH₂Cl₂/ether (1:2)(30 mL) then with ether (30 mL) and dried under reduced pressure. When purified in this manner, 4-chloro-3-methyl-5-(2-(2-(6-methylbenzo[d][1,3]dioxol-5-yl)acetyl)-3-thienylsulfonamido)isoxazole, sodium salt was obtained in high yield (5.7 g, 68%) with good purity (98.2% pure by HPLC). The product can also be further purified by recrystallization from EtOH/methyl t-butylether (MTBE) after the above procedure if the initial purity is sufficiently high.

### C. N-(4-Chloro-3-methyl-5-isoxazolyl)-2-[3,4-(methylenedioxy)-6-methyl]phenyl-acetyl-3-thiophenesulfonamide, sodium hydrogen phosphate salt also designated 4-Chloro-3-methyl-5-(2-(2-(6-methylbenzo[d][1,3]dioxol-5-yl)acetyl)-3-thienylsulfonamido)isoxazole, sodium hydrogen phosphate salt

To a solid mixture of n-(4-chloro-3-methyl-5-isoxazolyl)-2-[3,4-(methylenedioxy)-6-methyl]phenylacetyl-3-thiophenesulfonamide (1.1492 g, 2.5263 mmol) and sodium phosphate dibasic (0.3486 g, 2.5263 mmol) was added de-ionized water (25 ml) and acetonitrile (25 ml). The resulting mixture was well shaken and warmed at 50° C. to obtain a clear solution, which was filtered. The filtrate was frozen at −78° C. and lyophilized to give the salt as a yellow powder (≈1.50 g).

### Example 2

### Lyophilized Formulations

Lyophilized formulations were prepared by the protocol in Tables 1 and 2 below.

TABLE 1
Sitaxsentan Sodium Lyophilized Formulation

| | Quantity in a 10 mL vial |
|---|---|
| Component | (mg/vial) |
| Sitaxsentan Sodium | 250.0 |
| Sodium Citrate Dihydrate | 53.5 |
| L-Ascorbic Acid | 20.0 |
| D-Mannitol | 200.0 |
| Sodium Bisulfite | 66.0 |
| Sodium Sulfite | 20.0 |
| Sodium Hydroxide or Hydrochloride Acid | QS to pH 6 |

TABLE 2
Lyophilization Conditions

| Steps | Conditions |
|---|---|
| Step 1 | Loading vials on shelf set to 5° C. |
| Step 2, Freezing | Cool shelf to −40° C. |
| Step 3, Freezing | Hold at −40° C. for 4 hours |
| Step 4, Evacuation | Evacuate chamber to a pressure of 150 mtorr |
| Step 5, Primary | Heat shelf to −15° C., hold pressure at 150 mtorr |
| Drying | |
| Step 6, Primary | Hold at −15° C. and 150 mtorr for 50 hours |
| Drying | |
| Step 7, Secondary | Heat shelf to +25° C. and 50 mtorr |
| Drying | |
| Step 8, Secondary | Hold at +25° C. and 50 mtorr for a minimum of |
| Drying | 6 hours |

### Example 3

### Lyophilized Formulations

Three formulations of sitaxsentan sodium at 25 mg/mL containing ascorbic acid or monothioglycerol were prepared as follows:

Sitaxsentan Sodium Lyophilized Formulation

| | Quantity in a 10 mL vial |
|---|---|
| Component | (mg/vial) |
| Sitaxsentan Sodium | 250.0 |
| Sodium Citrate Dihydrate | 57.1 |
| L-Ascorbic Acid | 40.0 |
| D-Mannitol | 400.0 |
| Citric Acid Monohydrate | 1.3 |
| Sodium Hydroxide or Hydrochloride Acid | QS to pH 6.8 |

Sitaxsentan Sodium Lyophilized Formulation

| | Quantity in a 10 mL vial |
|---|---|
| Component | (mg/vial) |
| Sitaxsentan Sodium | 250.0 |
| Sodium Phosphate Dibasic Heptahydrate | 26.8 |
| L-Ascorbic Acid | 40.0 |
| D-Mannitol | 400.0 |
| Sodium Phosphate Monobasic | 13.9 |
| Monohydrate | |
| Sodium Hydroxide or Hydrochloride Acid | QS to pH 6.8 |

The formulations were lyophilized according to lyophilization cycle as follows: The batch was frozen to −45° C. The vacuum was started and controlled at 30 microns and then the shelf temperature was warmed to +20° C. over 10 hours and then held there until the cycle was competed based on moisture of the batch.

### Example 4

### Sitaxsentan 100 mg Coated Tablets

The tablets were manufactured on a one kg scale. The granulating solution was prepared by dissolving sodium phosphate, mono- and di-basic, and disodium EDTA in purified water. Ascorbyl palmitate was added to the sitaxsentan sodium drug substance and blended in a bag by hand for approximately 30 seconds. Approximately half of the microcrystalline cellulose was added to the bag and blended for an additional 30 seconds. The mixture was screened through a screen. The remaining intragranular components (i.e., remaining microcrystalline cellulose, lactose, HPMC, sodium starch glycolate) were screened through a screen and added to the mixture. The powders were then charged into a heated Glatt GPCG-1. The granulating solution was applied to the intragranular powders. Additional water was sprayed, if necessary, to achieve a visually desirable granulation. After that, the granulation was dried until an LOD of less than 2% was achieved. The dried granulation was milled through a Fitzmill with a 0.0024-sized screen. Extragranular components were screened and blended with the milled granulation in an 8-qt. V-blender for five minutes. Magnesium stearate was screened then blended with the mixture for three minutes. The final blends were compressed on a tablet press to 500 mg core tablets using 0.2900″×0.6550″ modified oval tooling.

Coating suspension was prepared by adding Sepifilm LP014 and Sepisperse Dry 3202 (Yellow) to water with mixing. Mixing continued until a homogenous suspension is formed. The tablets were coated using a Compu-lab coater with a 19″ coating pan.

TABLE 3
Sitaxsentan Sodium 100 mg Tablet Formulation

| Component | mg/tablet | % w/w |
|---|---|---|
| Sitaxsentan sodium | 100.0 | 20.0 |
| Microcrystalline Cellulose (Avicel PH 102) | 175.0 | 35.0 |
| Lactose Monohydrate Fast Flo (intragranular) | 84.3 | 16.9 |
| Lactose Monohydrate Fast Flo (extragranular) | 82.0 | 16.4 |
| Hydroxypropyl Cellulose E-5P | 25.0 | 5.0 |
| Ascorbyl Palmitate | 1.0 | 0.2 |
| EDTA, Disodium | 1.0 | 0.2 |
| Sodium Phosphate, Monobasic Monohydrate | 0.6 | 0.1 |
| Sodium Phosphate, Dibasic Anhydrous | 1.1 | 0.2 |
| Sodium Starch Glycolate (intragranular) | 12.5 | 2.5 |
| Sodium Starch Glycolate (extragranular) | 12.5 | 2.5 |
| Magnesium Stearate, Non-Bovine | 5.0 | 1.0 |
| Purified Water, USP | 192.5 | — |
| Total Core Tablet Weight | 500.0 | 100.0 |
| Sepisperse Dry 3202 (Yellow) | 8.0 | 1.6 |
| Sepifilm LP 014 | 12.0 | 2.4 |
| Total Coated Tablet Weight | 520.0 | 104.0 |

Since modifications will be apparent to those of skill in this art, it is intended that this invention be limited only by the scope of the appended claims.

What is claimed is:

1. A method for treatment or amelioration of one or more symptoms of sleep apnea, comprising administering a compound that is an endothelin antagonist to a patient in need of the treatment.

2. The method of claim 1, wherein the compound is selected from BE-18257B; BQ-123; PD 156707; L-754,142; SB 209670; SB 217242; A-127722; TAK-044; bosentan; sitaxsentan, and a pharmaceutically acceptable derivative thereof.

3. A method of claim 1, wherein the compound is selected from sitaxsentan and a pharmaceutically acceptable salt thereof.

4. The method of claim 1, wherein the compound is sitaxsentan.

5. The method of claim 3, wherein the compound is an alkali metal salt of sitaxsentan.

6. The method of claim 5, wherein the compound is sitaxsentan sodium.

7. The method of claim 1, wherein the sleep apnea is selected from obstructive sleep apnea and central sleep apnea.

8. The method of claim 3, wherein the compound is administered in a single dose.

9. The method of claim 8, wherein the compound is administered once daily.

10. The method of claim 3, wherein the compound is administered in an amount from about 20 mg up to about 350 mg/day.

11. The method of claim 10, wherein the amount of the compound administered is about 25 mg/day.

12. The method of claim 10, wherein the amount of the compound administered is about 50 mg/day.

13. The method of claim 10, wherein the amount of the compound administered is about 90 mg/day.

14. The method of claim 10, wherein the amount of the compound administered is about 100 mg/day.

15. The method of claim 10, wherein the amount of the compound administered is about 150 mg/day.

16. The method of claim 10, wherein the amount of the compound administered is about 300 mg/day.

17. The method of claim 10, wherein the compound is administered as an oral formulation.

18. The method of claim 17, wherein the oral formulation is a tablet.

19. The method of claim 18, wherein the tablet further comprises an antioxidant, a binding agent, a diluent, a buffer and a moisture resistant coating.

20. The method of claim 18, wherein the tablet further comprises microcrystalline cellulose, lactose monohydrate fast flo (intragranular), lactose monohydrate fast flo (extragranular), hydroxypropyl methylcellulose E-5P, ascorbyl palmitate, disodium EDTA, sodium phosphate monobasic, monohydrate, sodium phosphate dibasic, anhydrous, sodium starch glycoloate (intragranular), sodium starch glycoloate (extragranular) phosphate, magnesium stearate and a coating of Sepifilm LP014/Sepisperse Dry 3202 Yellow.

21. The method of claim 18, wherein the tablet comprises about 20% sitaxsentan sodium, about 35% microcrystalline cellulose, about 16.9% lactose monohydrate fast flo (intragranular), about 16.4% lactose monohydrate fast flo (extragranular), about 5.0% hydroxypropyl methylcellulose E-5P, about 0.2% ascorbyl palmitate, about 0.2% disodium (EDTA), about 0.1% sodium phosphate monobasic, monohydrate, about 0.2% sodium phosphate dibasic, anhydrous, about 2.5% sodium starch glycoloate (extragranular), about 2.5% sodium starch glycoloate (intragranular) phosphate, about 1% magnesium stearate, a coating of Sepifilm LPO 14/Sepisperse Dry 3202 Yellow at about 2.4%/1.6% weight gain.

22. The method of claim 18, wherein the tablet comprises about 100 mg sitaxsentan sodium, about 1.0 mg ascorbyl palmitate, about 1.0 mg disodium edetate (EDTA), about 25 mg hydroxypropyl methylcellulose E-5P, about 84.3 lactose monohydrate fast flo (intragranular), about 82 mg lactose monohydrate fast flo (extragranular), about 175 mg microcrystalline cellulose, about 0.6 mg sodium phosphate monobasic, monohydrate, about 1.1 mg sodium phosphate dibasic, anhydrous, about 12.5 mg sodium starch glycoloate (extragranular), about 12.5 mg sodium starch glycoloate (intragranular) phosphate, about 5 mg magnesium stearate, non-bovine and a coating of Sepifilm LP014 at about 12 mg and Sepisperse Dry 3202 Yellow at 8 mg.

23. The method of any of claims 1-16, wherein the compound is administered as a lyophilized powder.

24. The method of any of claims 1-16, wherein the lyophilized powder further comprises an antioxidant, a buffer and a bulking agent.

25. The method of any of claims 1-16, wherein the lyophilized powder comprises about 41% of sitaxsentan sodium, about 3.3% ascorbic acid, about 3.3% sodium sulfite and about 10.8% sodium bisulfite, about 8.8% sodium citrate dihydrate and about 32.8% mannitol.

26. The method of claim 23, wherein the lyophilized powder comprises about 33% of sitaxsentan sodium, about 5.3% ascorbic acid, about 7.6% sodium citrate dihydrate, about 53% D-mannitol and about 0.13% citric acid monohydrate by total weight of the lyophilized powder.

27. The method of claim 23, wherein the lyophilized powder comprises about 34% of sitaxsentan sodium, about 5.5% ascorbic acid, about 3.7% sodium phosphate dibasic heptahydrate, about 55% D-mannitol and about 1.9% sodium phosphate monobasic monohydrate by total weight of the lyophilized powder.

28. An article of manufacture comprising packaging material and a compound that is an endothelin antagonist, contained within the packaging material, wherein the packaging material includes a label that indicates that the compound is used for treating sleep apnea.

29. The article of manufacture of claim 28, wherein the compound is sitaxsentan sodium.
